(19) United States
(12) Patent Application Publication
(10) Pub. No.: US 2005/0157888 A1
(43) Pub. Date: Jul. 21, 2005

(54) Electronic stethoscope with piezo-electrical film contact microphone

(75) Inventor: Paul Yang, Jhonghe City (TW)

Correspondence Address: TROXELL LAW OFFICE PLLC, 5205 LEESBURG PIKE, SUITE 1404, FALLS CHURCH, VA, 22041, US

(73) Assignee: Health & Life Co., Ltd.

(21) Appl. No.: 10/758,076

(22) Filed: Jan. 16, 2004

(51) Int. Cl. A61B 7/04

(52) U.S. Cl. 381/67

(57) ABSTRACT

The present invention discloses an electronic stethoscope with a Piezo-Electrical Film contact microphone comprising a stethoscope head with a Piezo-Electrical Film contact microphone inside, and the stethoscope head is electrically connected to a circuit and a microcontroller unit (MCU). The microcontroller unit is connected to a front-end operational amplifier (OP-amp) circuit, a wave filter circuit, and a transmit circuit, such that when the stethoscope is used, the weak sound signal received by contacting stethoscope head with a human body is sent to the OP amplifier. The amplified sound signal (such as heart sound and lung sound) selectively measured by the switch module is processed by the microcontroller unit and the wave filter. The filtered sound signal is sent to a transmit/receive circuit, so that the wave filter circuit can filter the noise of the sound signal produced by human bodies under the control of the microcontroller unit, and medical people can make correct diagnostics based on the correct sound received through the transmit/receive circuits.

### FIELD OF THE INVENTION

The present invention relates to an electronic stethoscope with a contact microphone, which uses an electric circuit to amplify a weak sound signal (such as heart sound or lung sound) produced by human bodies, so that the sound signal can be separated from other noises, and allows medical people to make correct diagnostics.

### BACKGROUND OF THE INVENTION

Stethoscope is the one of the oldest and most original diagnostic tools. As stethoscope had been invented for more than two centuries, substantial improvements to overcome the shortcomings of the traditional stethoscope were made by electronic methods. Since the structure of the stethoscope generally uses a long empty tube to pass biological sounds to a doctor's ear, and the resonance so produced distorts the sound. Furthermore, the long-distance travel of the sound also causes a significant loss to the intensity of the sound. Therefore, traditional stethoscope not only has limitations on its acoustic performance, but also requires doctors to pay more attention to its use and concentrate on listening to the tiny sound signals from different parts of the patient's body. Therefore, it usually requires doctors to have some experience on using this device for a correct diagnostic, since doctors may easily misjudge a case due to human factors. Such stethoscope is not good for its application at all.

### SUMMARY OF THE INVENTION

In view of the foregoing shortcomings of the prior-art stethoscope assembly, the inventor of this invention focused on the problem and started thinking of a way to improve and overcome its deficiencies and tried to find a reasonable method to solve the problem. After extensive researches and studies, the inventor finally invented an electronic stethoscope with a Piezo-Electrical Film contact microphone.

The primary objective of the present invention is to provide an electronic stethoscope with a Piezo-Electrical Film contact microphone comprising a stethoscope head with a Piezo-Electrical Film contact microphone inside, and the stethoscope head is electrically connected to a circuit and a microcontroller unit (MCU).

The microcontroller unit is connected to a front-end operational amplifier (OP-amp) circuit, a wave filter circuit, and a transmit circuit, such that when the stethoscope is used, the weak sound signal received by contacting stethoscope head to a patient's body is sent to the OP amplifier. The amplified sound signal (such as heart sound and lung sound) selectively measured by the switch module is processed by the microcontroller unit and the wave filter. The filtered sound signal is sent to a transmit/receive circuit, so that the wave filter circuit can filter the noise of the sound signal produced by human bodies under the control of the microcontroller unit, and medical people can make correct diagnostics based on the correct sound received through the transmit/receive circuits.

Another objective of this invention is to provide a contact-type electronic stethoscope having wired or wireless transmit/receive circuits.

A further objective of this invention is to provide contact-type electronic stethoscope with its microcontroller unit connected to a display device, such that the diagnostic results is shown on the display device.

### BRIEF DESCRIPTION OF THE DRAWINGS

FIG. 1 is a perspective view of the stethoscope according to the present invention.

FIG. 2 is a block diagram of the circuit according to a preferred embodiment of the present invention.

FIG. 3 is a schematic circuit diagram of a preferred embodiment of the present invention.

FIG. 4 is a block diagram of another preferred embodiment of the present invention.

FIG. 5 is a schematic circuit diagram of another preferred embodiment of the present invention.

FIG. 6 is a view illustrating the application of the stethoscope according to the present invention.

### DETAILED DESCRIPTION OF THE PREFERRED EMBODIMENTS

To make it easier for our examiner to understand the objective of the invention, its structure, innovative features, and performance, we use a preferred embodiment together with the attached drawings for the detailed description of the invention.

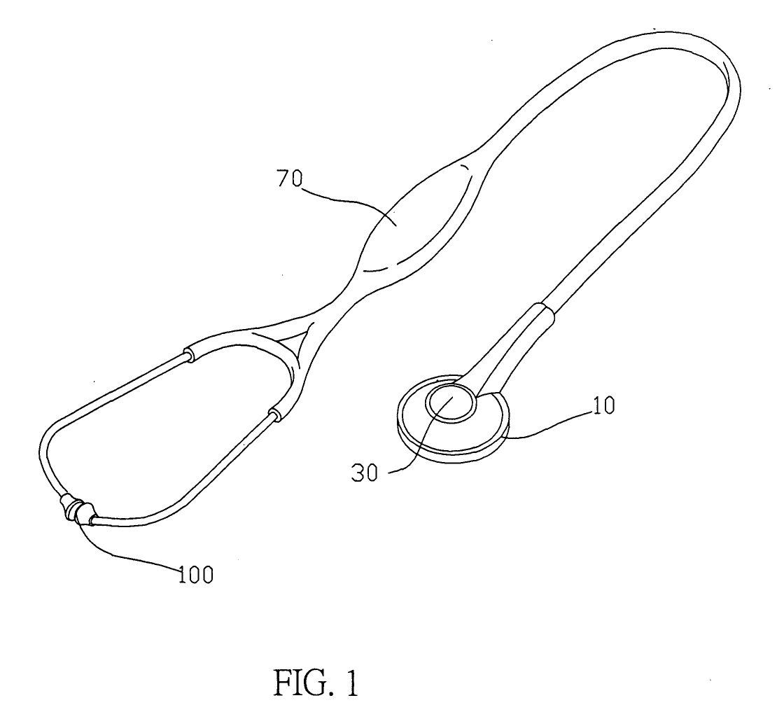

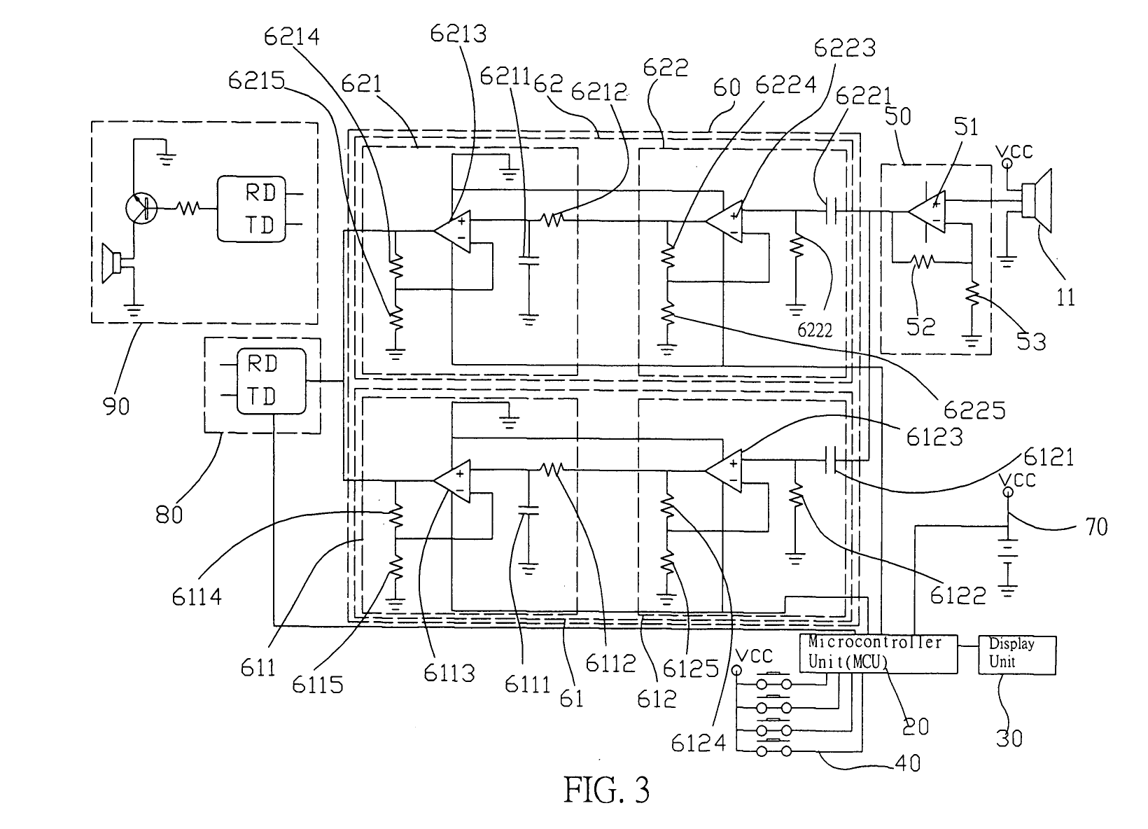

Please refer to FIGS. 1, 2 and 3 for the electronic stethoscope with a Piezo-Electrical Film contact microphone according to the present invention comprising a stethoscope head 10 with a Piezo-Electrical Film contact microphone 11 inside, and the stethoscope head 10 is electrically connected to a circuit and a microcontroller unit (MCU) 20. The MCU chips used in this embodiment is TMP86CM23U. The microcontroller unit 20 is connected to a display unit 30 and a switch module 40 by an electric circuit, wherein the display unit is an LCD monitor, and the switch module 40 has one or more switches for switching between the heart sound mode and the lung sound mode.

Further, the stethoscope head 10 is connected to a front-end operational amplifier (OP-amp) circuit 50, and the OP amplifier 50 comprises an amplifier 51 and a resistor 52, 53 to define a feedback circuit. This embodiment adopts SANYO LA6324N as the amplifier 51, wherein the input end of the amplifier 51 is connected to the Piezo-Electrical Film contact microphone, and the output end of the amplifier 51 is connected to a wave filter circuit 60. The wave filter circuit 60 is mainly used for filtering noises. Since different sound signals have specific frequencies, noises with frequency other than the specific frequency of the sound signal are filtered, and the sound signal with specific frequency remains. The wave filter circuit 60 is connected to the microcontroller unit 20 and the wave filter circuit 60 comprises a heart sound wave filter 61 and a lung sound wave filter 62. The heart sound wave filter 61 and the lung sound wave filter 62 respectively comprise a low-pass wave filter 611, 621 and a high-pass wave filter 612, 622. The low-pass wave filter 611, 621 and the high-pass wave filter 612, 622 respectively comprise a capacitor 6111, 6211, 6121, 6221. The capacitors 6111, 6211, 6121, 6221 are connected respectively to a resistor 6112, 6212, 6122, 6222 to filter the frequencies other than the specified one, and the capacitors 6111, 6211, 6121, 6221 are connected respectively to an amplifier circuit, and the amplifier circuit comprises an amplifier 6113, 6213, 6123, 6223, and a resistor 6114, 6115, 6214, 6215, 5215, 6124, 6125, 6224, 6225. The amplifier used in this embodiment is SANYO LA6324N.

Further, the microcontroller unit (MCU) 20 is connected to a power supply 70 and a transmit circuit 80 by a circuit, wherein the power supply 70 can be either alternate current or direct current, and the transmit circuit 80 used in this embodiment is a Bluetooth module. However, the persons skilled in the art can still use other wireless module (such as an infrared) to substitute the Bluetooth module, so that the microcontroller unit 20 can work together with a wireless receive circuit 90 by the transmit circuit 80. The processed sound signal is sent directly to the receive circuit 90 without going through the electric circuit. The wireless receive circuit 90 of this embodiment is a Bluetooth receive module, and the receive circuit 90 is installed in an electronic product (such as a wireless earphone, a PDA, or a computer, etc) so that medical people can receive the diagnostic result by connecting to the electronic product with the wireless receive circuit 90. The diagnostic result can be saved for future follow-ups and observations.

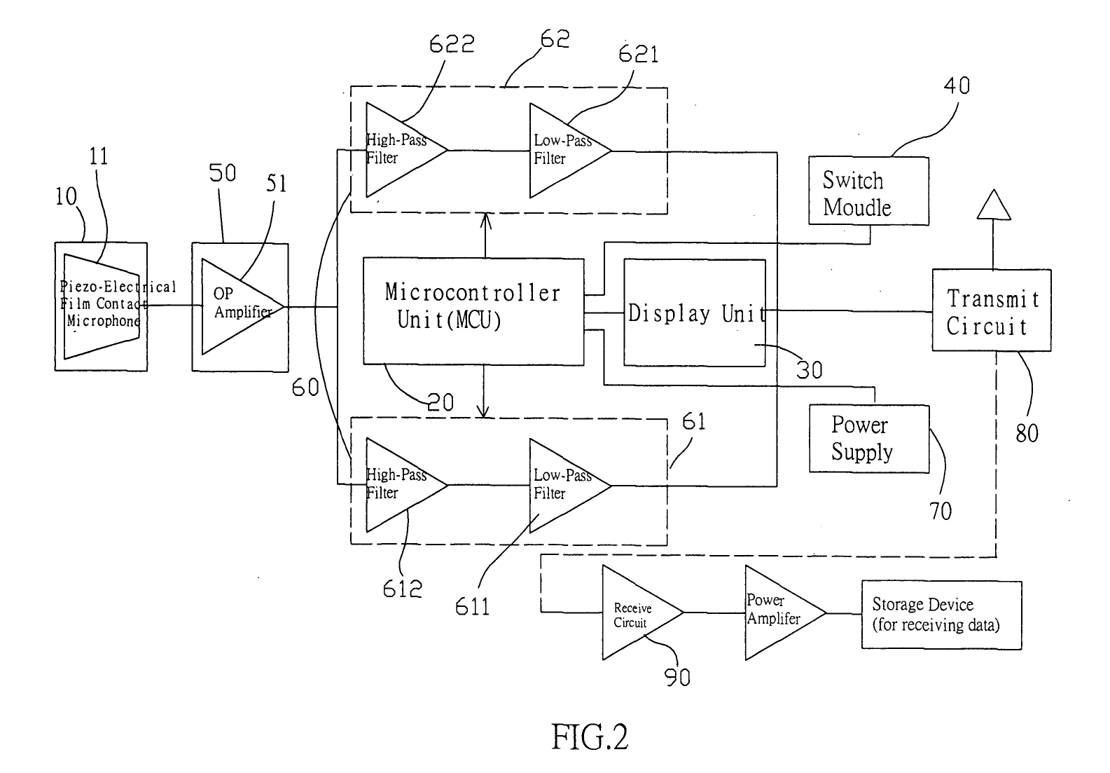

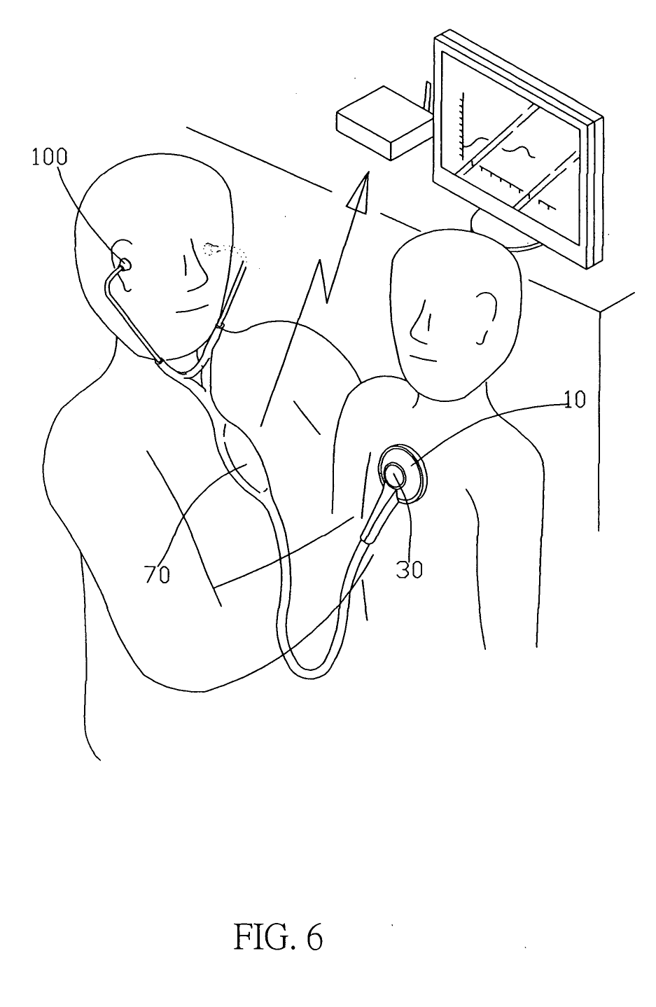

Please refer to FIGS. 2, 3, and 6. When the stethoscope is in use and the stethoscope head 10 is in contact with the patient's body, the control of the microcontroller unit 20 will send the weak sound signal produced by the patient's body to the OP amplifier circuit 50. After the sound signal is amplified and processed, the microcontroller unit 20 will send the sound signal by determining the measuring mode (such as heart sound mode or lung sound mode) selected by the switch module 40 to a designated wave filter for the processing, and the result of the processed sound signal is shown on a display unit 30. In the meantime, the transmit circuit 80 sends the processed sound signal to the receive circuit 90, so that the sound signal (such as heart sound or lung sound) produced by the patient's body allows medical people to make correct diagnostics. The result of such sound signal is recorded and saved for future follow-ups and observations.

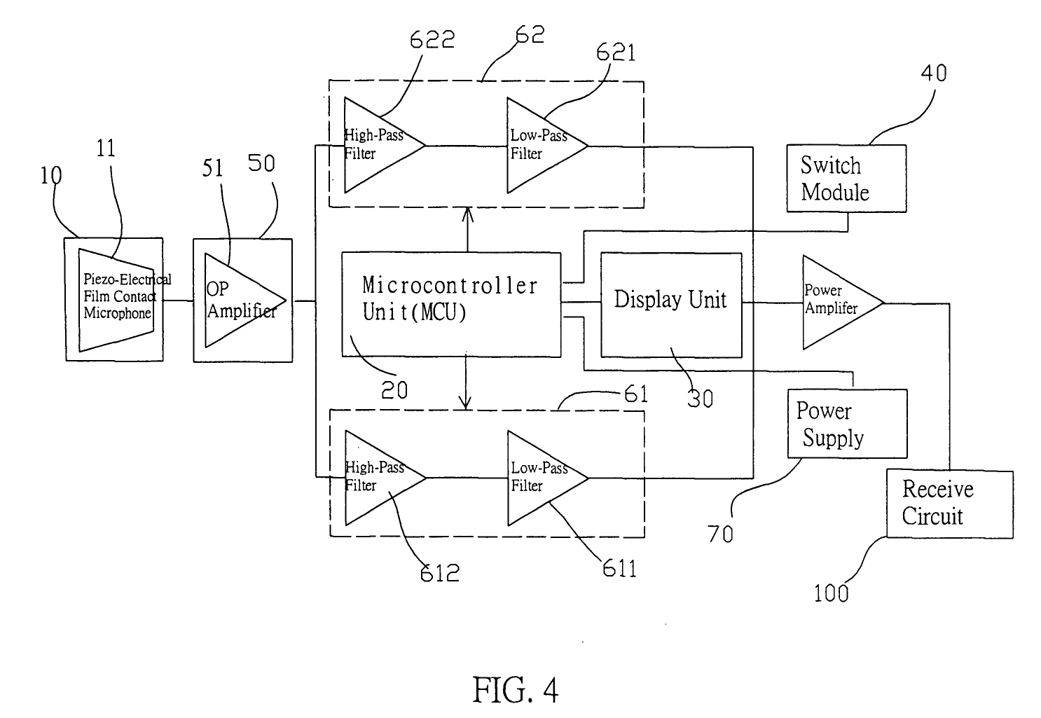

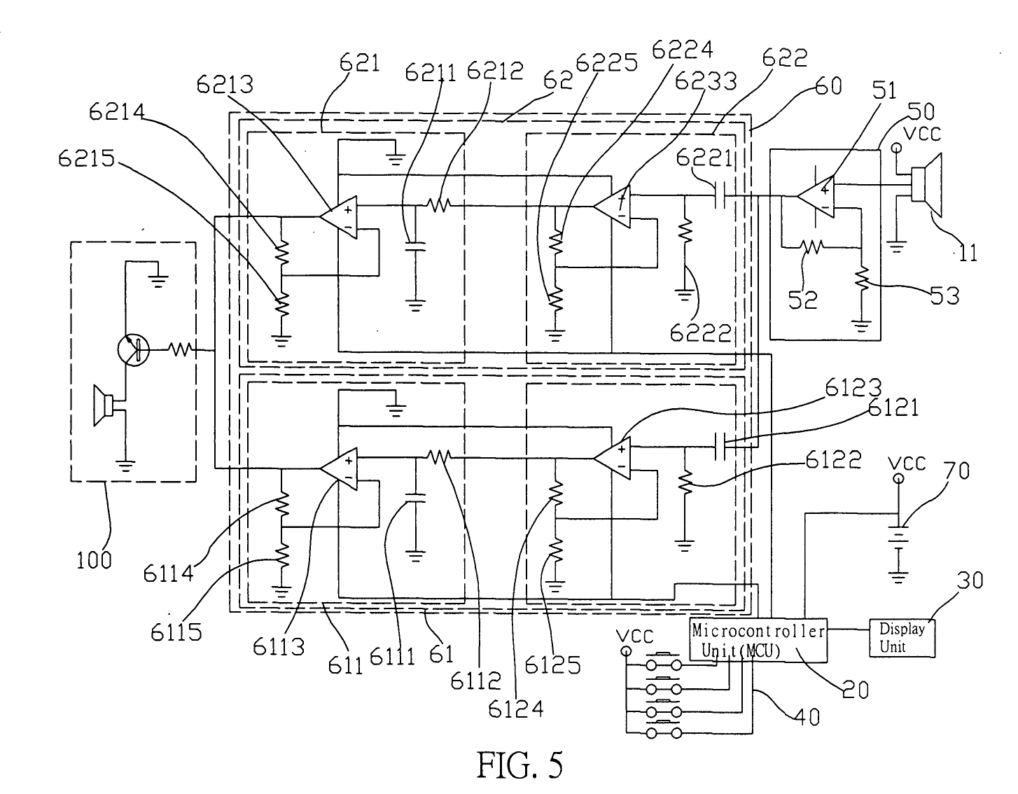

Further, please refer to FIGS. 1, 4, and 5. The stethoscope of the present invention comprises a stethoscope head 10, and the stethoscope head 10 has a Piezo-Electrical Film contact microphone 11, and the stethoscope head 10 is connected to a microcontroller unit (MCU) 20 by an electric circuit. This embodiment adopts the TMP86CM23U for the MCU chip. The microcontroller unit 20 is connected to a display unit 30 and a switch module 40 by an electric circuit, wherein the display unit is an LCD monitor, and the switch module 40 has one or more switches for switching the heart sound mode or the lung sound mode.

Further, the stethoscope head 10 is connected to a front-end operational amplifier (OP-amp) circuit 50, and the OP amplifier 50 comprises an amplifier 51 and the anode of the amplifier 51 is connected to the Piezo-Electrical Film contact microphone 11, and the cathode of the OP amplifier 51 is connected with a resistor 52, 53. The OP amplifier circuit 50 is connected to a wave filter circuit 60 by an electric circuit, and the wave filter circuit 60 is mainly used for filtering noises. Since different sound signals have specific frequencies, noises with frequency other than the specific frequency of the sound signal are filtered, and the sound signal with specific frequency remains. The wave filter circuit 60 is connected to the microcontroller unit 20 and the wave filter circuit 60 comprises a heart sound wave filter 61 and a lung sound wave filter 62. The heart sound wave filter 61 and the lung sound wave filter 62 respectively comprise a low-pass wave filter 611, 621 and a high-pass wave filter 612, 622. The low-pass wave filter 611, 621 and the high-pass wave filter 612, 622 respectively comprise a capacitor 6111, 6211, 6121, 6221. The capacitors 6111, 6211, 6121, 6221 are connected respectively to a resistor 6112, 6212, 6122, 6222 to filter the frequencies other than the specified one, and the capacitors 6111, 6211, 6121, 6221 are connected respectively to an amplifier circuit, and the amplifier circuit comprises an amplifier 6113, 6213, 6123, 6223, and a resistor 6114, 6115, 6214, 6215, 5215, 6124, 6125, 6224, 6225. The amplifier used in this embodiment is SANYO LA6324N.

Further, the microcontroller unit (MCU) 20 is connected to a power supply 70 and a transmit circuit 80 by a circuit, wherein the power supply 70 can be either alternate current or direct current for supply power for driving the components, and the receive circuit used in this embodiment is an electronic earphone.

Please refer to FIGS. 1, 4, 5, and 6. When the stethoscope is in use and the stethoscope head 10 is in contact with the patient's body, the control of the microcontroller unit 20 will send the weak sound signal produced by the patient's body to the OP amplifier circuit 50. After the sound signal is amplified and processed, the microcontroller unit 20 will base on the switch signal sent from the switch module 40 to determine which wave filter (such as the heart sound wave filter or the lung sound wave filter) in the wave filter circuit 60 should be used for the processing. The filtered and processed sound signal is sent to the receive circuit 100, so that the sound signal produced by the patient's body is filtered by the wave filter under the control of the microcontroller to remove any noise and the correct sound signal is received through the receive circuit (such as an electronic earphone) by medical people to make correct diagnostics.

In summation of the above description, the present invention discloses a better and operable electronic stethoscope and enhances the performance of the conventional structure, and further complies with the patent application requirements and is submitted to the Patent and Trademark Office for review and granting of the commensurate patent rights.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures

1. An electronic stethoscope with Piezo-Electrical Film contact microphone, comprising:
a stethoscope head, having a Piezo-Electrical Film contact microphone thereon;
a OP amplifier circuit, electrically coupled to said stethoscope head, for amplifying a sound signal received by said Piezo-Electrical Film contact microphone and converting the sound signal into a current;
a wave filter circuit, electrically coupled to said OP amplifier circuit, and comprising a heart sound wave filter and a lung sound wave filter, for filtering any noise with a frequency other than that of a specified sound and keeping the specified sound;
a microcontroller unit (MCU), electrically coupled to said OP amplifier circuit and wave filter circuit, for controlling the actions of said OP amplifier circuit and wave filter circuit;
a receive circuit, electrically coupled to said microcontroller unit, for receiving a signal after being filtered and processed;
a switch module, electrically coupled to said microcontroller unit, for setting a measuring mode selected from the collection of a heart sound mode and a lung sound mode; and
by means of foregoing elements, the sound signal received by contacting said stethoscope head onto a patient's body being sent to said OP amplifier circuit, and the amplified sound signal being sent to a wave filter specified by said wave filter circuit according to the measuring mode selected by said switch module, and the filtered sound signal being sent to said receive circuit, so that medial people being capable of making correct diagnostics after the noise of the sound signal being filtered by said wave filter circuit under the control of said microcontroller unit.

2. The electronic stethoscope with Piezo-Electrical Film contact microphone of claim 1, wherein said microcontroller unit is electrically coupled to a display unit for displaying the result.

3. The electronic stethoscope with Piezo-Electrical Film contact microphone of claim 2, wherein said display unit is a liquid crystal display.

4. The electronic stethoscope with Piezo-Electrical Film contact microphone of claim 1, wherein said receive circuit is a wireless receive circuit.

5. The electronic stethoscope with Piezo-Electrical Film contact microphone of claim 4, wherein said wireless receive circuit is a wireless electronic earphone.

6. The electronic stethoscope with Piezo-Electrical Film contact microphone of claim 1, wherein said receive circuit is a wired receive circuit.

7. The electronic stethoscope with Piezo-Electrical Film contact microphone of claim 6, wherein said wired circuit is a wired electronic earphone.
